(19) United States
(12) Patent Application Publication
(10) Pub. No.: US 2026/0274862 A1
(43) Pub. Date: Sep. 17, 2026

(54) METHOD FOR PRODUCING ß-LACTAM COMPOUND

(71) Applicant: FUJIFILM Corporation, Tokyo (JP)

(72) Inventors: Seiichi HITOMI, Kanagawa (JP); Kenji WADA, Kanagawa (JP)

(73) Assignee: FUJIFILM Corporation, Tokyo (JP)

(21) Appl. No.: 19/667,626

(22) Filed: May 4, 2026

Related U.S. Application Data
(63) Continuation of application No. PCT/JP2024/044151, filed on Dec. 13, 2024.

(30) Foreign Application Priority Data
Dec. 14, 2023 (JP) 2023-211441

(51) Int. Cl. C07D 499/12; C07D 499/68; C07D 501/04

(52) U.S. Cl. CPC C07D 499/12; C07D 499/68; C07D 501/04

(57) ABSTRACT

There is provided a method for producing a β-lactam compound, the method including: introducing, into different flow channels, each of
a reaction solution (i) obtained by dissolving a metal salt (y) represented by Formula (2) generated by introducing, in a solution of a metal salt (x) represented by Formula (1), a protective group into an amino group of the metal salt (x),
a solution (ii) of an acid halide, and
a solution (iii) of a β-lactam compound (a) having an amino group, and causing each solution to flow in each flow channel;
merging the reaction solution (i) and the solution (ii) to generate a mixed acid anhydride; and
merging the merged solution (M1) and the solution (iii) to generate a β-lactam compound (b) having an amide bond,

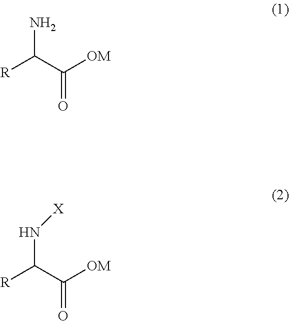

in the formulae, R represents a cyclic hydrocarbon group, M represents an alkali metal, and X represents a protective group.

### CROSS-REFERENCE TO RELATED APPLICATIONS

This application is a Continuation of PCT International Application No. PCT/JP2024/044151 filed on Dec. 13, 2024, which claims priority under 35 U.S.C. § 119 (a) to Japanese Patent Application No. 2023-211441 filed in Japan on Dec. 14, 2023. Each of the above applications is hereby expressly incorporated by reference, in its entirety, into the present application.

### BACKGROUND OF THE INVENTION

### 1. Field of the Invention

The present invention relates to a method for producing a β-lactam compound.

### 2. Description of the Related Art

A β-lactam compound (a compound having a β-lactam ring) is widely known as an antibiotic or the like, and is an important compound for maintaining the health of humans and other animals. As a β-lactam antibiotic, for example, ampicillin, epicillin, cephalexin, cefradine, cefaclor, and the like are known.

As a method for synthesizing these β-lactam compounds, a so-called Dane salt method is known. For example, Patent Document 1 describes a method for synthesizing ampicillin by a Dane salt method. In this method, 4-picoline is added as a catalyst to a suspension containing a high content of D-N-(1-ethoxycarbonylprop-2-yl)-α-aminophenyl potassium acetate, which is a Dane salt, in n-butyl acetate, and the mixture is stirred at room temperature for 15 hours and then cooled to −33° C. Next, pivaloyl chloride is added thereto, and the mixture is stirred at −20° C. for 90 minutes to react with the Dane salt, thereby obtaining a mixture containing a mixed carboxylic acid anhydride. Separately from this, triethylamine (TEA) as a base is added to a mixed solvent of isopropanol (IPA) and water, and a solution in which 6-aminopenicillanic acid (6-APA) is dissolved in this liquid is prepared. This solution is added dropwise to the above-described mixture at a temperature of −45° C. to −30° C. for 45 minutes, and the reaction mixture is further stirred at −30° C. to −35° C. for 90 minutes to form an amide bond between the mixed carboxylic acid anhydride and 6-APA, and water acts to eliminate a 1-ethoxycarbonylprop-2-yl group (also known as a 3-ethoxy-1-methyl-3-oxo-1-propenyl group) which is a protective group, thereby producing ampicillin.

The reaction is shown as a reaction scheme as follows.

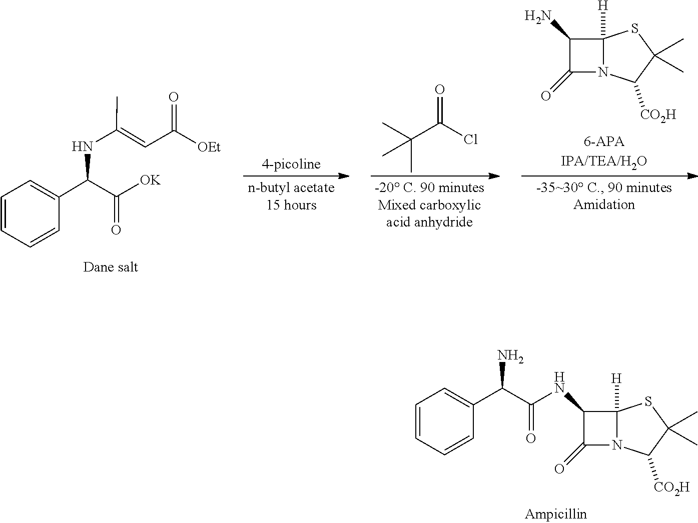

### SUMMARY OF THE INVENTION

The above-described Dane salt has extremely low solubility in a solvent, and in a case where the Dane salt is used in a reaction in a solution state, the concentration is too low, which is not practical for industrial production. Therefore, at present, the Dane salt has to be used in a reaction in a suspension state as described above. Therefore, the synthesis of the β-lactam compound by the Dane salt method is practically limited to a batch-type reaction, and as described above, a relatively long reaction time is required while mixing the suspension.

In a chemical synthesis reaction, a flow-type reaction capable of continuous synthesis is known, unlike a batch-type reaction. In the flow-type reaction, in general, two or more kinds of raw material solutions are caused to flow in different flow channels, the solutions are combined downstream, and a target synthesis reaction proceeds while the merged solution further flows downstream. Therefore, the target product can be continuously obtained as long as the raw material supply is continued, and precise temperature control of the reaction system, improvement of stirring efficiency by combining the liquids, and the like are combined, which makes it possible to increase the production efficiency as compared with the batch-type reaction. However, the flow-type reaction is a reaction system in which the liquid smoothly flows in the flow channel. In a case where a suspension containing a high content of the above-described Dane salt is applied to the flow-type reaction, the liquid cannot smoothly flow in the flow channel, and the flow channel or the like is blocked during the flow, which is not practical.

An object of the present invention is to provide a method for producing a β-lactam compound, which makes it possible to continuously obtain a target β-lactam compound with high efficiency through a flow-type reaction, from a starting raw material having low solubility in a solvent, such as a Dane salt, via a generation of a mixed acid anhydride to an amidation reaction.

In view of the above-described problems, the present inventors have conducted intensive studies. As a result, the present inventors have found that, in a case where a glycin compound having a cyclic hydrocarbon group, such as phenylglycine, is dissolved in an organic solvent in which an alkali metal hydroxide is dissolved to be in a state of an alkali metal salt, and a protective group is introduced into an amino group of the metal salt to synthesize a Dane salt or a similar salt, the salt into which the protective group is introduced has low solubility in a solvent as described above in a solid state, but the salt is in a state of being dissolved at a high concentration in a synthesis reaction solution, and even in a case where the synthesis reaction solution is concentrated to some extent, the salt can still maintain a dissolved state.

The present invention has been further studied based on these findings, and has been completed.

The above-described objects of the present invention have been achieved by the following methods.

[1]
A method for producing a β-lactam compound, the method comprising: introducing, into different flow channels, each of
a reaction solution (i) obtained by dissolving a metal salt (y) represented by Formula (2) generated by introducing, in a solution obtained by dissolving a metal salt (x) represented by Formula (1), a protective group into an amino group of the metal salt (x),
a solution (ii) obtained by dissolving an acid halide, and
a solution (iii) obtained by dissolving a β-lactam compound (a) having an amino group, and causing each solution to flow in each flow channel;
merging the reaction solution (i) and the solution (ii) to cause the metal salt (y) and the acid halide to react with each other in a merged solution (M1) while the merged solution (M1) flows downstream to generate a mixed acid anhydride; and
merging the merged solution (M1) and the solution (iii) to cause the mixed acid anhydride and the β-lactam compound (a) having an amino group to react with each other in a merged solution (M2) while the merged solution (M2) flows downstream to generate a β-lactam compound (b) having an amide bond,

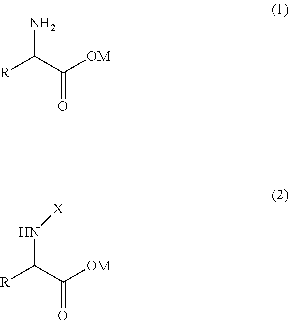

in the formulae, R represents a cyclic hydrocarbon group, M represents an alkali metal, and X represents a protective group.
   [2]

The method for producing a β-lactam compound according to [1], in which the reaction solution (i) and the solution (ii) are merged at −40° C. to 0° C.

[3]

The method for producing a β-lactam compound according to [1] or [2], in which the merged solution (M1) and the solution (iii) are merged at −40° C. to 0° C.

[4]

The method for producing a β-lactam compound according to any one of [1] to [3], in which the solution (iii) contains 5% to 50% by mass of water.

[5]

The method for producing a β-lactam compound according to any one of [1] to [4], in which the solution (iii) contains a base.

[6]

The method for producing a β-lactam compound according to any one of [1] to [5], in which the protective group is a 3-ethoxy-1-methyl-3-oxo-1-propenyl group and/or a 3-methoxy-1-methyl-3-oxo-1-propenyl group.

[7]

The method for producing a β-lactam compound according to any one of [1] to [6], in which the acid halide includes a carboxylic acid halide.

[8]

The method for producing a β-lactam compound according to [7], in which a reaction between the metal salt (y) and the carboxylic acid halide in the merged solution (M1) is performed in the presence of a basic catalyst.

[9]

The method for producing a β-lactam compound according to [8], in which the basic catalyst has a pyridine skeleton.

[10]

The method for producing a β-lactam compound according to [8] or [9], in which a molar amount of the basic catalyst is set to 0.001 to 0.1 with respect to a molar amount of 1 of the β-lactam compound (a) having an amino group.

[11]

The method for producing a β-lactam compound according to any one of [7] to [10], in which the carboxylic acid halide includes a carboxylic acid chloride.

[12]

The method for producing a β-lactam compound according to any one of [1] to [11], in which a hydrocarbon ring of the cyclic hydrocarbon group is a benzene ring or a 1,4-cyclohexadiene ring.

[13]

The method for producing a β-lactam compound according to any one of [1] to [12], in which M is sodium or potassium.

[14]

The method for producing a β-lactam compound according to any one of [1] to [13], in which the β-lactam compound (a) having an amino group is 6-aminopenicillanic acid or 7-amino-desacetoxycephalosporanic acid.

[15]

The method for producing a β-lactam compound according to any one of [1] to [14], in which the β-lactam compound (b) is ampicillin.

In the present specification, a numerical value range expressed using “to” means a range that includes the preceding and succeeding numerical values of “to” as the lower limit value and the upper limit value, respectively.

In the present invention, a term representing a substituent has a meaning including not only the substituent itself but also a form in which the substituent further has a substituent, as long as the effect of the present invention is not impaired. For example, the term “cyclic hydrocarbon group” has a meaning including both an unsubstituted cyclic hydrocarbon group and a cyclic hydrocarbon group having a substituent. In addition, for example, the term “phenyl” has a meaning including both an unsubstituted phenyl group and a phenyl group having a substituent.

With the method for producing a β-lactam compound according to the embodiment of the present invention, even in a case of using a starting material having low solvent solubility such as a Dane salt, a target β-lactam compound can be continuously obtained with high efficiency through a flow-type reaction from the generation of a mixed acid anhydride to an amidation reaction.

### BRIEF DESCRIPTION OF THE DRAWINGS

FIG. 1 is a schematic view showing an example of a flow reactor system used in a method for producing a β-lactam compound according to the embodiment of the present invention.

### DESCRIPTION OF THE PREFERRED EMBODIMENTS

### [Method for Producing β-Lactam Compound]

In the method for producing a β-lactam compound according to the embodiment of the present invention (hereinafter, also referred to as “the method for producing a β-lactam compound of the present invention”), a flow-type reaction is adopted. In the flow-type reaction, the following reaction solution (i), solution (ii), and solution (iii) are caused to flow in different flow channels, and the reaction solutions are sequentially combined to proceed with a target reaction.

### Reaction Solution (i):

Reaction solution (i): a reaction solution obtained by dissolving a metal salt (y) represented by Formula (2) generated by introducing a protective group into an amino group of a metal salt (x) represented by Formula (1) in a solution obtained by dissolving the metal salt (x).

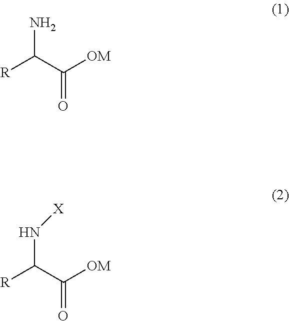

In the formulae, R represents a cyclic hydrocarbon group, M represents an alkali metal, and X represents a protective group.

The metal salt (x) represented by Formula (1) may be an L-form or a D-form, and for example, from the viewpoint of exhibiting higher antimicrobial properties, the metal salt (x) is preferably a D-form, which is a final product, a β-lactam compound (b) described later. The same applies to the metal salt (y) represented by Formula (2).

### Solution (ii):

Solution (ii): a solution obtained by dissolving an acid halide.

Solution (iii):

Solution (iii): a solution obtained by dissolving a β-lactam compound (a) having an amino group.

In the above-described flow-type reaction, by combining the reaction solution (i) and the solution (ii), the metal salt (y) and the acid halide react with each other to generate a mixed acid anhydride while the merged solution (M1) flows downstream. Further, the merged solution (M1) and the solution (iii) are combined, and the mixed acid anhydride and the β-lactam compound (a) having an amino group react with each other while the merged solution (M2) flows downstream, to generate a β-lactam compound (b) having an amide bond. The β-lactam compound (b) having an amide bond is a target β-lactam compound (final product) to be produced by the production method according to the embodiment of the present invention.

In the present specification, the terms “upstream” and “downstream” are used for a direction in which a liquid flows, and a side into which the liquid is introduced (a side from which the liquid flows) is upstream, and a side from which the liquid flows out is downstream.

Preferred aspects of the reaction solution (i), the solution (ii), and the solution (iii) will be described.

<Reaction Solution (i)>

As described above, the reaction solution (i) is a reaction solution obtained by dissolving the metal salt (y) generated by introducing a protective group into the amino group of the metal salt (x) (by a protective group introduction reaction) in a solution obtained by dissolving the metal salt (x). That is, the reaction solution itself in which the protective group introduction reaction has occurred, or a concentrated solution or a diluted solution of the reaction solution can be used as the reaction solution (i). In the reaction solution (i), the metal salt (y) is maintained in a solution state without being precipitated. From the viewpoint of reaction efficiency, the reaction solution (i) is preferably the concentrated solution. The reaction solution (i) is preferably capable of maintaining a solution state at −20° C., more preferably capable of maintaining a solution state at −30° C., still more preferably capable of maintaining a solution state at −40° C., and even still more preferably capable of maintaining a solution state at −50° C.

For the protective group introduction reaction, an example of a reaction scheme will be shown below using a reaction (that is, one aspect of the synthesis reaction of the Dane salt) in which D-phenylglycine is used as a starting material and a 3-ethoxy-1-methyl-3-oxo-1-propenyl group is introduced as a protective group of an amino group. In the following scheme, Et represents ethyl.

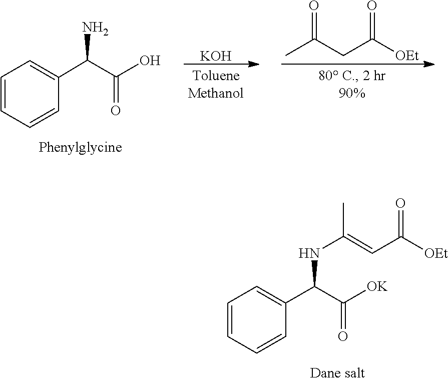

In the above reaction scheme, D-phenylglycine is in a potassium salt state in a mixed solvent of toluene and methanol by potassium hydroxide. This potassium salt corresponds to the metal salt (x). Next, the potassium salt is reacted with ethyl acetoacetate to introduce a protective group into the amino group of the potassium salt, thereby obtaining a Dane salt. This Dane salt corresponds to the metal salt (y). Methanol is used for a part of the solvent in the above reaction scheme in order to dissolve potassium hydroxide. Since methanol may cause a side reaction in the subsequent reaction, it is preferable that the reaction solution (i) is used as a concentrated solution obtained by removing methanol from the above-described reaction solution.

Hereinafter, preferred aspects of the reaction solution (i) in the present invention will be described without being limited to the above-described exemplary reaction scheme.

In the metal salt (x), as represented by Formula (1), in a compound in which a carbon atom to which an amino group of glycine is bonded has a cyclic hydrocarbon group as a substituent, the carboxy group is in an alkali metal salt state. The hydrocarbon ring (cyclic hydrocarbon) of the cyclic hydrocarbon group is preferably a 6-membered ring, and more preferably a benzene ring or a 1,4-cyclohexadiene ring. In addition, the hydrocarbon ring may be unsubstituted, or may have a substituent as long as the effect of the present invention is not impaired. Preferred specific examples of the metal salt (x) include a compound in which the carboxy group is in a salt state by an alkali metal in phenylglycine, 2-(2,5-dihydroxyphenyl)glycine, 4-fluoro-2-phenylglycine, 2-(2-chlorophenyl)glycine, and 2-(4-chlorophenyl)glycine.

The metal salt (x) is a salt of an alkali metal, and is preferably a sodium salt or a potassium salt, and more preferably a potassium salt.

The protective group to be introduced in the above-described protective group introduction reaction is not particularly limited as long as it is a protective group of an amino acid. Examples thereof include an Ac group (acetyl group), a Boc group (t-butoxycarbonyl group), a Cbz group (carbobenzoxy group), an Fmoc group (9-fluorenylmethyloxycarbonyl group), a Bz group (benzoyl group), a Bzl group (benzyl group), a Troc group (2,2,2-trichloroethoxycarbonyl group), a Teoc group (2-(trimethylsilyl)ethoxycarbonyl group), an Alloc group (allyloxycarbonyl group), a 3-ethoxy-1-methyl-3-oxo-1-propenyl group, and a 3-methoxy-1-methyl-3-oxo-1-propenyl group. From the viewpoint that the protective group can be easily deprotected with water contained in the solution (iii), the protective group to be introduced in the above-described protective group introduction reaction is preferably a 3-ethoxy-1-methyl-3-oxo-1-propenyl group or a 3-methoxy-1-methyl-3-oxo-1-propenyl group, and more preferably a 3-ethoxy-1-methyl-3-oxo-1-propenyl group.

The solvent for the above-described protective group introduction reaction is not particularly limited as long as it can dissolve an alkali metal compound (preferably an alkali metal hydroxide) for forming a salt with an alkali metal, can dissolve the metal salt (x), and can dissolve the metal salt (y). As such a solvent, a mixed solvent of an aromatic hydrocarbon solvent and an aliphatic alcohol solvent is preferable. It is preferable that the respective solvents constituting the mixed solvent are compatible with each other (do not undergo phase separation).

Examples of the aromatic hydrocarbon solvent include benzene, toluene, styrene, o-xylene, p-xylene, m-xylene, and mesitylene. One kind or two or more kinds of aromatic hydrocarbon solvents can be used as the aromatic hydrocarbon solvent.

As the aliphatic alcohol solvent, for example, a lower alcohol such as methanol, ethanol, or propanol is preferable. One kind or two or more kinds of aliphatic alcohol solvents can also be used in combination.

In the mixed solvent of the aromatic hydrocarbon solvent and the aliphatic alcohol solvent, a mixing ratio of each solvent is not particularly limited and can be appropriately set. For example, a proportion of the aliphatic alcohol solvent in the mixed solvent can be 1% to 40% by mass, more preferably 3% to 30% by mass, and still more preferably 5% to 20% by mass. By containing the aliphatic alcohol solvent in the above-described preferred amount, a base such as a hydroxide of an alkali metal can be sufficiently dissolved, the above-described metal salt (x) can be generated with high efficiency in the mixed solvent, and the metal salt (x) can be present in a dissolved state.

From the viewpoint of further suppressing a side reaction in the subsequent reaction, it is preferable that the reaction solution (i) to be caused to flow in the flow channel in the flow-type reaction is obtained by removing the aliphatic alcohol solvent from the reaction solution of the above-described protective group introduction reaction. That is, the solvent constituting the reaction solution (i) is preferably an aromatic hydrocarbon solvent. From the viewpoint of achieving both improvement of productivity and precipitation suppression by increasing the concentration, a concentration of solid contents (concentration of components other than the solvent) in the reaction solution (i) is preferably 10% to 40% by mass, more preferably 10% to 30% by mass, and still more preferably 20% to 30% by mass. For example, as described above, the solvent solubility of the solid Dane salt is extremely low. However, the reaction solution (i) is a solution in which the metal salt (y) is dissolved at a concentration significantly higher than that of the sea salt.

A proportion of the metal salt (y) in the solid contents in the reaction solution (i) is preferably 50% by mass or more, more preferably 60% by mass or more, still more preferably 70% by mass or more, and even still more preferably 80% by mass or more. It is preferable that all the solid contents in the reaction solution (i) are the metal salt (y), but a certain amount of by-products may be generated or raw materials may remain. The proportion of the metal salt (y) in the solid contents in the reaction solution (i) is preferably 50% to 100% by mass, and it is also preferably 50% to 98% by mass, 60% to 97% by mass, 70% to 96% by mass, and 80% to 95% by mass.

In the present invention, in a case where the metal salt (y) obtained by the above-described protective group introduction reaction is precipitated to be in a solid state (dried product), and the solid state metal salt (y) is dissolved in a mixed solvent of toluene/methanol=90/10 (mass ratio), a solubility at 25° C. (an amount of the metal salt (y) dissolved in 100 g of the mixed solvent in a case where 100 g of the mixed solvent and 10 g of the solid state metal salt (y) are mixed and caused to stand at 25° C. for 24 hours) is preferably 1.0 g or less. For example, the above-described Dane salt satisfies the requirement of the solubility.

The reaction solution (i) preferably contains a basic catalyst. The basic catalyst catalyzes the subsequent amidation reaction with the β-lactam compound (a) having an amino group. Examples of the basic catalyst include 4-dimethylaminopyridine (DMAP), 4-picoline, N-methylimidazole, 2,6-lutidine, N-methylmorpholine, 1,4-diazabicyclo[2.2.2]octane (DABCO), N-benzyldimethylamine, N-methylpiperidine, and dicyclohexylmethylamine.

The basic catalyst is preferably a compound having a tertiary amino group, more preferably a compound having a pyridine skeleton or a compound having an imidazole skeleton, and still more preferably a compound having a pyridine skeleton. Among the above-described specific examples, DMAP, 4-picoline, and/or N-methylimidazole can be suitably used, and DMAP is particularly preferable.

The molar amount of the basic catalyst is preferably used to be 0.001 to 0.1 with respect to 1 molar amount of the β-lactam compound (a) having an amino group.

<Solution (ii)>

The acid halide contained in the solution (ii) is preferably a carboxylic acid halide, a sulfonic acid halide, or a haloformate ester. In the present invention, the term “acid halide” is used in a broader sense than usual. More specifically, in the present invention, the term “acid halide” is used to include a haloformate ester in addition to a general acid halide.

The halogen atom in the acid halide is preferably a chlorine atom. Among these, the acid halide is preferably a carboxylic acid halide and more preferably a carboxylic acid chloride. Preferred specific examples of the acid halide include pivaloyl chloride (PivCl), methanesulfonyl chloride, and ethyl chloroformate, and from the viewpoint of improving reactivity and suppressing by-products, pivaloyl chloride can be suitably used.

As the solvent of the solution (ii), the aromatic hydrocarbon solvent described in the reaction solution (i) is suitable. Examples thereof include benzene, toluene, styrene, o-xylene, p-xylene, m-xylene, and mesitylene, and one or two or more of these can be used. It is preferable that the aromatic hydrocarbon solvent used in the solution (ii) and the aromatic hydrocarbon solvent used in the reaction solution (i) are the same type.

The concentration of the acid halide in the solution (ii) is not particularly limited, and for example, it can be appropriately set based on the stoichiometry in consideration of the amount of the metal salt (y) in the mixed solution (merged solution (M1)) obtained by combining the reaction solution (i) and the solution (ii), and also in consideration of the flow rate of the reaction solution (i) and the solution (ii). For example, the concentration of the acid halide in the solution (ii) can be set to 5% to 25% by mass, and it is also preferably set to 10% to 20% by mass. In order to more reliably convert the metal salt (y) into the mixed acid anhydride, it is preferable that an acid halide of 1.05 equivalents or more is present with respect to the metal salt (y) immediately after the reaction solution (i) and the solution (ii) are combined (assuming that the merged solution is still unreacted). In addition, from the viewpoint of suppressing the reaction of the acid halide with the β-lactam compound (a) having an amino group in the solution (iii) to be reacted thereafter, it is preferable that an acid halide of 1.50 equivalents or less is present with respect to the metal salt (y) immediately after the reaction solution (i) and the solution (ii) are combined. It is more preferable that an acid halide of 1.05 to 1.30 equivalents (preferably 1.05 to 1.20 equivalents) is present with respect to the metal salt (y) immediately after the reaction solution (i) and the solution (ii) are combined.

<Solution (iii)>

The β-lactam compound (a) having an amino group, which is contained in the solution (iii), is not particularly limited as long as it is a β-lactam compound having an amino group. In a case where the merged solution (M1) and the solution (iii) are combined, the mixed acid anhydride (activated carboxy group) and the amino group of the β-lactam compound (a) having an amino group react with each other while the merged solution (M2) is flowing downstream, and a β-lactam compound (b) having an amide bond, which is a target product, is generated.

From the viewpoint of obtaining a β-lactam compound (b) useful as an antibiotic or the like, preferred specific examples of the β-lactam compound (a) having an amino group include 6-aminopenicillanic acid (6-APA), 7-amino-desacetoxycephalosporanic acid (7-ADCA), and 7-amino-3-chlorocephalosporanic acid (7-ACCA) having the following structures.

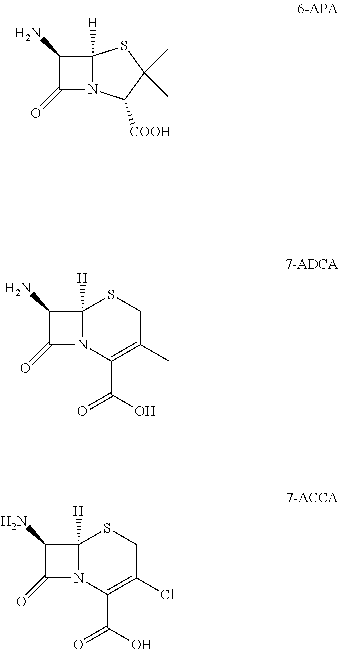

By using 6-APA as the β-lactam compound (a) having an amino group, it is possible to obtain, for example, ampicillin or epicillin having the following structure as the β-lactam compound (b). In addition, by using 7-ADCA as the β-lactam compound (a) having an amino group, it is possible to obtain, for example, cephalexin or cefradine having the following structure as the β-lactam compound (b). Further, by using 7-ACCA as the β-lactam compound (a) having an amino group, it is possible to obtain, for example, cefaclor having the following structure.

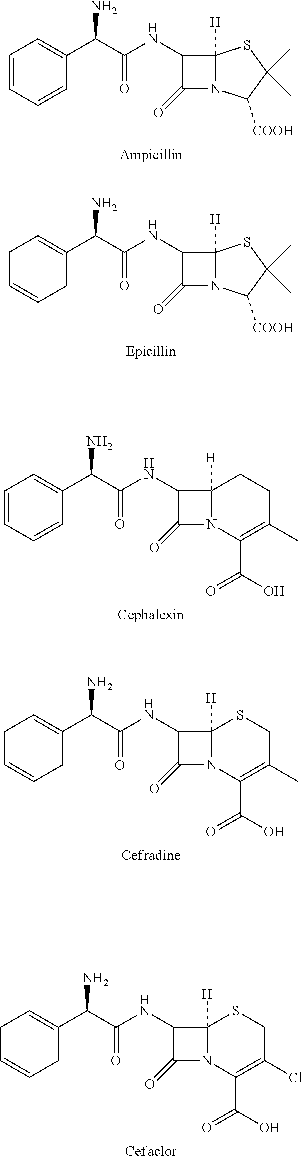 [SMILES: CC1(C)S[C@@H]2C(NC(=O)[C@H](N)c3ccccc3)C(=O)N2[C@H]1C(=O)O ; CC1(C)S[C@@H]2C(NC(=O)[C@H](N)C3=CCC=CC3)C(=O)N2[C@H]1C(=O)O ; CC1=C(C(=O)O)N2C(=O)C(NC(=O)[C@H](N)c3ccccc3)[C@H]2CC1 ; CC1=C(C(=O)O)N2C(=O)C(NC(=O)[C@H](N)C3=CCC=CC3)[C@H]2SC1 ; N[C@@H](C(=O)NC1C(=O)N2C(C(=O)O)=C(Cl)CS[C@H]12)C1=CCC=CC1]

As the solvent of the solution (iii), for example, a solvent obtained by mixing an organic solvent and water is preferable. In addition, the solution (iii) preferably contains a base. By containing a base, the solubility of the β-lactam compound (a) having an amino group can be further enhanced. In addition, by containing water, it is also possible to deprotect the above-described protective group without requiring a special operation.

The organic solvent is not particularly limited, and examples thereof include a water-soluble organic solvent compatible with water (for example, an alcohol, acetone, acetonitrile, N,N-dimethylacetamide, tetrahydrofuran, and the like). From the viewpoint of further suppressing by-products, the above-described organic solvent is preferably an aliphatic alcohol solvent, more preferably an aliphatic lower alcohol solvent having 1 to 3 carbon atoms, and still more preferably isopropyl alcohol.

From the viewpoint of further enhancing the solubility of the β-lactam compound (a) having an amino group, the content of water in the solution (iii) is preferably 5% to 50% by mass, more preferably 8% to 40% by mass, and still more preferably 10% to 30% by mass.

Examples of the above-described base include an aliphatic amine, an aromatic amine, and a heterocyclic amine. Among these, a trialkylamine is preferable, and triethylamine can be particularly suitably used. By containing a base, the pH of the solution (iii) is preferably controlled to 7.5 to 9.5 and more preferably controlled to 8.0 to 9.0. As a result, the solubility of the β-lactam compound (a) having an amino group can be further enhanced.

The content of the β-lactam compound (a) having an amino group in the solution (iii) is not particularly limited, and can be appropriately set based on the stoichiometry in consideration of the amount of the mixed acid anhydride in the mixed solution (merged solution (M2)) immediately after the solution (iii) is combined with the merged solution (M1), and also in consideration of the flow rate or the like of the merged solution (M1) and the solution (iii). For example, the content of the β-lactam compound (a) having an amino group in the solution (iii) can be set to 5% to 25% by mass, and it is also preferably set to 10% to 20% by mass.

Immediately after the merged solution (M1) and the solution (iii) are combined (assuming that the merged solution is still unreacted), it is preferable to have 0.7 to 1.3 equivalents of the β-lactam compound (a) having an amino group with respect to the mixed acid anhydride, and it is more preferable to have 0.8 to 1.0 equivalents of the β-lactam compound (a) having an amino group.

### <Flow Reactor System>

An embodiment of a flow reactor system (flow reactor system) used in the present invention will be described with reference to the drawings. Each drawing is an explanatory view for facilitating the understanding of the present invention, and the size, the relative size relationship, or the like of each member may be changed for convenience of description, and does not show the actual relationship as it is. In addition, external shapes or shapes other than those defined by the present invention are not limited to those shown in the drawing.

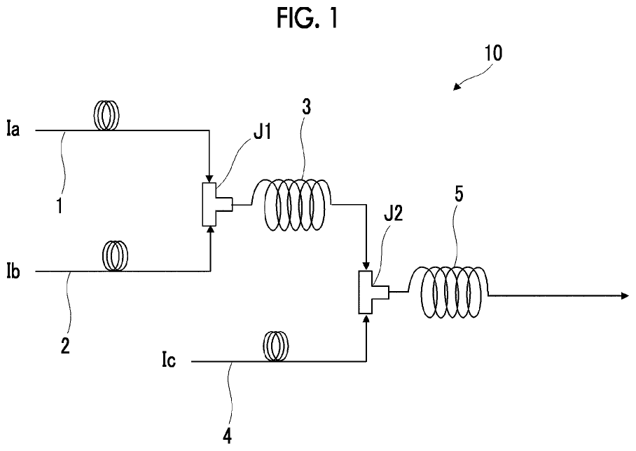

FIG. 1 is a schematic view showing an example of a flow reactor system used in the manufacturing method according to the embodiment of the present invention. The flow reactor system (10) shown in FIG. 1 comprises a flow channel (1) comprising an introduction port (Ia) for introducing the above-described reaction solution (i), a flow channel (2) comprising an introduction port (Ib) for introducing the solution (ii), a merging section (J1) where the flow channel (1) and the flow channel (2) are merged, a reaction flow channel (3) connected to a downstream end portion of the merging section (J1), a flow channel (4) provided with an introduction port (Ic) for introducing the above-described solution (iii), a merging section (J2) where the reaction flow channel (3) and the flow channel (4) are merged, and a reaction flow channel (5) connected to a downstream end portion of the merging section (J2).

Each of the introduction ports (Ia), (Ib), and (Ic) is usually connected to a liquid feeding pump (not shown) such as a syringe pump or a diaphragm pump, and by operating this pump, each solution can be circulated in each flow channel at a desired flow rate.

Each configuration of the embodiment shown in FIG. 1 will be described in more detail.

### (Flow Channel (1))

The flow channel (1) is a flow channel that supplies the reaction solution (i) introduced from the introduction port (Ia) to the merging section (J1). The equivalent diameter of the flow channel (1) is preferably 0.2 to 50 mm. By setting the equivalent diameter of the flow channel (1) to 0.2 mm or more, it is possible to suppress an increase in pressure during liquid feeding, and it is also possible to suppress the blockage of the flow channel even in a case where insoluble matter is generated. In addition, by setting the equivalent diameter of the flow channel (1) to 50 mm or less, the liquid temperature during the introduction into the merging section (J1) can be appropriately controlled. The equivalent diameter of the flow channel (1) is more preferably 0.5 to 30 mm and still more preferably 1 to 20 mm.

“Equivalent diameter” is a term used in the field of mechanical engineering, and is also called hydraulic equivalent diameter. Assuming that there is a circular tube equivalent to a pipe line or a flow channel having a given inner cross-sectional shape of the tube, the diameter of the inner cross-section of the equivalent circular tube is called equivalent diameter. The equivalent diameter (deq) is defined as deq=4A/p in which A represents an inner cross-sectional area of a pipe line and p represents a wetted perimeter (inner perimeter) of a pipe line. In a case where the above definition is applied to a circular tube, the equivalent diameter equals the diameter of the inner cross section of the circular tube. Based on the data regarding an equivalent circular tube, the equivalent diameter is used for estimating the fluidity or the heat transfer characteristics of the pipe line, and shows the spatial scale (representative length) of a phenomenon. For a square tube in which a represents one side of the inner cross section of the tube, the equivalent diameter dep=4a²/4a=a; for an equilateral triangular tube in which a represents one side thereof, deq=a/3^{1/2}; and for a flow between parallel flat plates in which h represents a height of a flow channel, deq=2h (for example, see “Mechanical Engineering Dictionary”, edited by The Japan Society of Mechanical Engineers, 1997, Maruzen Co., Ltd).

The length of the flow channel (1) is not particularly limited, and the flow channel (1) can be configured with, for example, a tube having a length of about 10 cm to 200 m (preferably 30 cm to 100 m).

The material of the tube is not particularly limited, and examples thereof include perfluoroalkoxy alkane (hereinafter, referred to as PFA), polytetrafluoroethylene (hereinafter, referred to as PTFE), an aromatic polyether ketone-based resin, stainless steel, copper or a copper alloy, nickel or a nickel alloy, titanium or a titanium alloy, quartz glass, and lime soda glass. From the viewpoint of flexibility and chemical resistance, the material of the tube is preferably PFA, PTFE, stainless steel, a nickel alloy, or titanium.

The flow rate at which the reaction solution (i) is introduced from the introduction port (Ia) is not particularly limited, and can be appropriately set according to the purpose in consideration of the equivalent diameter of each flow channel, the concentration of each liquid, the introduction flow rate, and the like. For example, the flow rate is preferably 0.1 to 10,000 mL/min (per minute), more preferably 0.5 to 8,000 mL/min, and still more preferably 1 to 6,000 mL/min.

The temperature of the flow channel (1) can be set to, for example, −40° C. to 0° C., and is preferably −30° C. to −10° C. and more preferably −25° C. to −15° C.

### (Flow Channel (2))

The flow channel (2) is a flow channel that supplies the solution (ii) introduced from the introduction port (Ib) to the merging section (J1). The equivalent diameter of the flow channel (2) is preferably 0.2 to 50 mm. By setting the equivalent diameter of the flow channel (2) to 0.2 mm or more, it is possible to suppress an increase in pressure during liquid feeding, and it is possible to suppress the blockage of the flow channel even in a case where insoluble matter is generated. In addition, by setting the equivalent diameter of the flow channel (2) to 50 mm or less, the liquid temperature during the introduction into the merging section (J1) can be appropriately controlled. The equivalent diameter of the flow channel (2) is more preferably 0.5 to 30 mm and still more preferably 1 to 20 mm.

The length of the flow channel (2) is not particularly limited, and the flow channel (2) can be configured with, for example, a tube having a length of about 10 cm to 200 m (preferably 30 cm to 100 m).

The material of the tube is not particularly limited, and a tube made of the material exemplified in the flow channel (1) can be used.

The flow rate at which the solution (ii) is introduced from the introduction port (Ib) is not particularly limited, and can be appropriately set according to the purpose in consideration of the equivalent diameter of each flow channel, the concentration of each liquid, the introduction flow rate, and the like. For example, the flow rate is preferably 0.1 to 10,000 mL/min (per minute), more preferably 0.5 to 8,000 mL/min, and still more preferably 1 to 6,000 mL/min.

In addition, a relationship between a flow rate rB at which the solution (ii) is introduced from the introduction port (Ib) and a flow rate rA at which the liquid (i) is introduced from the introduction port (Ia) is not particularly limited, and can be appropriately set in consideration of the concentration of each solution and the like. For example, [flow rate rA]/[flow rate rB] can be set to 10/1 to 1/10, and is preferably 5/1 to 1/5 and more preferably 3/1 to 1/3. In the present specification, the unit of a flow rate is mL/min.

The temperature of the flow channel (2) can be set to, for example, −40° C. to 0° C., and is preferably −30° C. to −10° C. and more preferably −25° C. to −15° C.

### <Merging Section (J1)>

The reaction solution (i) introduced into the flow channel (1) is combined with the solution (ii) flowing in the flow channel (2) at the merging section (J1). The merging section (J1) has a role of a mixer, and is not particularly limited as long as the flow channel (1) and the flow channel (2) can be combined into one flow channel and the liquid combined with the reaction flow channel (3) connected to the downstream end part of the merging section (J1) can be sent out.

In the embodiment of FIG. 1, a T-shaped connector having three connection ports is used as the merging section (J1). From the viewpoint of further improving the mixing performance, the equivalent diameter of the flow channel in the merging section (J1) is preferably 0.2 to 30 mm.

The material of the merging section (J1) is not particularly limited, and for example, a material consisting of PFA, PTFE, an aromatic polyether ketone-based resin, stainless steel, copper or a copper alloy, nickel or a nickel alloy, titanium or a titanium alloy, quartz glass, or lime soda glass can be used.

As the T-shaped connector, a commercially available product can be widely used, and for example, a cross connector manufactured by Upchurch Scientific, a union cross manufactured by Swagelok Company, a four-way joint manufactured by EYELA, or a SUS cross mixer manufactured by IDEX can be used. In the present invention, the merging section (J1) is not limited to the T-shaped connector, and for example, a Y-shaped connector may be used. In addition, a form may be adopted in which the reaction solution (i) and/or the solution (ii) is introduced from a plurality of introduction ports using a connector having four or more connection ports.

### (Reaction Flow Channel (3))

The reaction solution (i) and the solution (ii) are combined and mixed in the merging section (J1), and then the merged solution (M1) flows into the reaction flow channel (3). While the merged solution (M1) flows downstream in the reaction flow channel (3), the metal salt (y) dissolved in the reaction solution (i) reacts with the acid halide dissolved in the solution (ii) to generate a mixed acid anhydride. The generated mixed acid anhydride is present in the merged solution (M1) in a dissolved state. That is, the reaction flow channel (3) is a flow channel that produces a solution of the mixed acid anhydride and supplies the solution to the merging section (J2).

The form of the reaction flow channel (3) is not particularly limited, and a tube is usually used. The preferred material of the reaction flow channel (3) is the same as the preferred material of the flow channel (1) described above. In addition, the residence time of the merged solution in the flow channel (3) in the merging section (J1) can be adjusted by the equivalent diameter and the length of the reaction flow channel (3), the flow rate setting of the liquid feeding pump, and the like. The equivalent diameter of the reaction flow channel (3) is preferably 0.2 to 50 mm, more preferably 0.3 to 30 mm, still more preferably 0.5 to 20 mm, even more preferably 0.7 to 15 mm, and even still more preferably 1 to 12 mm. In addition, the length of the reaction flow channel (3) is preferably 0.5 to 50 m and more preferably 1 to 30 m. In order to more uniformly mix the reaction solution (i) and the solution (ii) in the reaction flow channel (3) and to more reliably obtain a solution of the mixed acid anhydride, a static mixer may be disposed in the middle of the reaction flow channel (3).

The residence time of the merged solution flowing in the reaction flow channel (3) is preferably 0.1 to 20 minutes, more preferably 0.2 to 15 minutes, and still more preferably 0.4 to 10 minutes.

The temperatures of the merging section (J1) and the reaction flow channel (3) can be set to, for example, −40° C. to 0° C., preferably −30° C. to −10° C., and more preferably −25° C. to −15° C.

<Flow channel (4)>

The flow channel (4) is a flow channel that supplies the solution (iii) introduced from the introduction port (Ic) to the merging section (J2). The equivalent diameter of the flow channel (4) is preferably 0.2 to 50 mm. By setting the equivalent diameter of the flow channel (4) to 0.2 mm or more, it is possible to suppress an increase in pressure during liquid feeding, and it is also possible to suppress the blockage of the flow channel even in a case where insoluble matter is generated. In addition, by setting the equivalent diameter of the flow channel (4) to 50 mm or less, the liquid temperature during the introduction into the merging section (J2) can be appropriately controlled. The equivalent diameter of the flow channel (4) is more preferably 0.5 to 30 mm and still more preferably 1 to 20 mm.

The length of the flow channel (4) is not particularly limited, and the flow channel (4) can be configured with, for example, a tube having a length of about 10 cm to 200 m (preferably 30 cm to 100 m).

The material of the tube is not particularly limited, and a tube made of the material exemplified in the flow channel (1) can be used.

The flow rate at which the solution (iii) is introduced from the introduction port (Ic) is not particularly limited, and can be appropriately set according to the purpose in consideration of the equivalent diameter of each flow channel, the concentration of each liquid, the introduction flow rate, and the like. For example, the flow rate is preferably 0.1 to 30,000 m/min (minute), more preferably 0.5 to 20,000 mL/min, and still more preferably 1 to 15,000 mL/min.

In addition, a relationship between a flow rate rD at which the merged solution (M1) is introduced from the reaction flow channel (3) into the merging section (J2) and a flow rate rC at which the solution (iii) is introduced from the introduction port (Ic) is not particularly limited, and can be appropriately set in consideration of the concentration of each solution and the like. For example, [flow rate rC]/[flow rate rD] can be set to 10/1 to 1/10, preferably 5/1 to 1/5, and more preferably 3/1 to 1/3.

The temperature of the flow channel (4) can be set to, for example, −40° C. to 0° C., preferably −30° C. to −10° C., and more preferably −25° C. to −15° C.

### <Merging Section (J2)>

The merged solution (M1) flowing in the reaction flow channel (3) and the solution (iii) flowing in the flow channel (4) are combined in the merging section (J2). The merging section (J2) has a role of a mixer, and is not particularly limited as long as the reaction flow channel (3) and the flow channel (4) can be combined into one flow channel and the liquid combined into the reaction flow channel (5) connected to the downstream end part of the merging section (J2) can be sent out.

In the embodiment of FIG. 1, a T-shaped connector having three connection ports is used as the merging section (J2). From the viewpoint of further improving the mixing performance, the equivalent diameter of the flow channel in the merging section (J2) is preferably 0.2 to 30 mm.

The material, shape, number of connection ports, and the like of the merging section (J2) can be applied as described in the merging section (J1).

### <Reaction Flow Channel (5)>

The merged solution combined and mixed in the merging section (J2) flows into the reaction flow channel (5), and the β-lactam compound (a) and the mixed acid anhydride react with each other to form an amide bond and generate the target β-lactam compound (b) while flowing downstream in the reaction flow channel (5).

The form of the reaction flow channel (5) is not particularly limited, and for example, a tube may be used. The preferred material of the reaction flow channel (5) is the same as the preferred material of the flow channel (1) described above. In addition, the reaction time can be adjusted by the equivalent diameter and length of the reaction flow channel (5), the flow rate setting of the liquid feeding pump, and the like. Usually, the equivalent diameter of the reaction flow channel (5) is preferably 0.2 to 50 mm, more preferably 0.3 to 30 mm, still more preferably 0.5 to 20 mm, even more preferably 0.7 to 15 mm, and even still more preferably 1 to 12 mm. In addition, the length of the reaction flow channel (5) is preferably 0.5 to 50 m and more preferably 1 to 30 m. In order to make the mixing of the merged solution (M1) and the solution (iii) more uniform in the reaction flow channel (5), a static mixer may be disposed in the middle of the reaction flow channel (5).

The residence time of the merged solution flowing in the reaction flow channel (5) is preferably 0.1 to 10 minutes, more preferably 0.2 to 8 minutes, and still more preferably 0.3 to 5 minutes.

The temperature of the merging section (J2) and the reaction flow channel (5)(that is, the reaction temperature) can be set to, for example, −40° C. to 0° C., and it is preferably −30° C. to −10° C. and more preferably −25° C. to −15° C.

In a case where a reaction solution in which the target β-lactam compound (b) is generated is taken out during the flow in the reaction flow channel (5), the target β-lactam compound (b) can be obtained in the taken-out reaction solution. In addition, the β-lactam compound (b) obtained in the reaction solution can be taken out as a solid by subjecting the β-lactam compound (b) to general crystallization, filtration, or the like. For example, by adding hydrochloric acid to the taken-out reaction solution to make the reaction solution acidic and extracting the β-lactam compound (b) into the water phase, and then adding a base such as ammonia water to bring the pH close to the neutral side, the β-lactam compound (b) can be precipitated in the water phase. By collecting the precipitate by filtration, a solid β-lactam compound (b) can be obtained. In addition, the impurities in the water phase can be removed by dissolving the collected β-lactam compound (b) by adding, for example, aqueous sodium bicarbonate and washing the water phase with ethyl acetate or the like. Next, by adjusting the pH and collecting the precipitated solid by filtration, a β-lactam compound (b) having a higher purity can be obtained.

The present invention will be described in more detail based on Examples; however, the present invention should not be construed as being limited to these Examples except as defined in the present invention.

### EXAMPLES

### Examples

<Preparation of Reaction Solution (i)>

A reaction solution (i) was obtained by synthesizing a Dane salt as a metal salt (y) through a potassium salt of D-phenylglycine as a metal salt (x) according to the following reaction scheme.

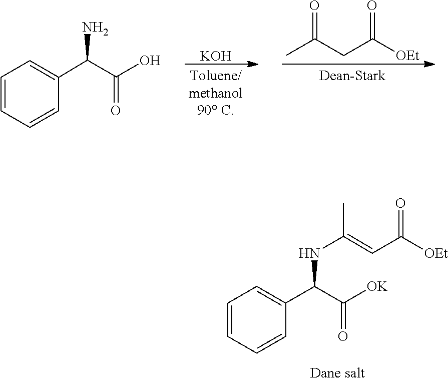

Toluene (450 mL), methanol (50 mL), D-phenylglycine (50 g), and KOH (20.2 g) were added to a 1 L three-neck flask, and the temperature was raised to 80° C. to 90° C. to dissolve the mixture, thereby obtaining a solution. Thereafter, ethyl acetoacetate (46.1 g) was added thereto, a Dean-Stark apparatus was attached thereto, and the mixture was stirred for 2 hours while maintaining the temperature at 80° C. to 90° C. The mixture was cooled to room temperature, insoluble matter was filtered off, and the obtained solution was attached to an evaporator and concentrated by distilling off the solvent. Toluene (200 mL) was added thereto at a time when the concentration of solid contents was about 30% by mass (about 30% by mass as the Dane salt concentration), and the mixture was concentrated again to obtain a toluene solution of the Dane salt (the concentration of solid contents was about 25% by mass, and the Dane salt concentration was about 25% by mass). Methanol was completely removed by the above-described concentration operation.

DMAP (0.0073 g) was added to the toluene solution (80.8 g) of the Dane salt to obtain a reaction solution (i).

<Preparation of Solution (ii)>

PivCl (14.0 g) was dissolved in toluene (70.0 g) to obtain a solution (ii).

<Preparation of Solution (iii)>

6-APA (12.0 g) was dissolved in a mixed solution of isopropyl alcohol (IPA, 56.1 g), water (12.0 g), and triethylamine (TEA, 7.1 g) to obtain a solution (iii).

### <Flow Reaction>

The production method according to the embodiment of the present invention was carried out as follows by a flow reaction.

A flow reactor system shown in FIG. 1 was installed in a constant-temperature tank at −20° C., and a flow reaction was carried out. Therefore, the liquid flowing in the flow channel and the merging section in this flow reactor system was −20° C.

The liquid feeding was carried out using a syringe pump.

As the flow channels (1) and (2), the reaction flow channel (3), the flow channel (4), and the reaction flow channel (5), a perfluoroalkoxy alkane (PFA) tube having an outer diameter of ⅛ inches and an inner diameter of 1.58 mm was used.

As the merging sections (J1) and (J2), a union tee (SS-200-3) manufactured by Swagelok Company was used.

The reaction solution (i) was introduced into the flow channel (1) from the introduction port (Ia) at a flow rate of 2.4 mL/min. In addition, the solution (ii) was introduced into the flow channel (2) from the introduction port (Ib) at a flow rate of 1.6 mL/min. In this way, the two solutions were combined at the merging section (J1), the merged solution (M1) was caused to flow into the reaction flow channel (3) on the downstream side, and a mixed acid anhydride was generated in the merged solution (M1). In the merged solution (assuming an unreacted uniform merged solution) immediately after the combination in the merging section (J1), about 1.09 equivalents of the acid halide were present with respect to the metal salt (y).

The solution (iii) was introduced from the introduction port (Ic) at a flow rate of 3.0 mL/min, the solution (iii) and the merged solution (M1) were combined at the merging section (J2), the merged solution (M2) was caused to flow into the reaction flow channel (5) on the downstream side, and the amidation reaction and the deprotection by the action of water were caused to proceed. In the merged solution (assuming an unreacted uniform merged solution) immediately after the combination in the merging section (J2), about 0.9 equivalents of the β-lactam compound (a) having an amino group was present with respect to the mixed acid anhydride.

The residence time of the merged solution (M1) in the reaction flow channel (3) was 7 minutes, and the residence time of the merged solution (M2) in the reaction flow channel (5) was 1 minute.

The reaction solution was sampled from the outlet of the reaction flow channel (5) over 20 minutes. 1.5 N hydrochloric acid (62 mL) was added to the sampled reaction solution, and ampicillin, which is the target β-lactam compound (b), was dissolved in the water phase. 28% by mass of ammonia water was added to the water phase to adjust the pH to 4.5 to 5.0, and the precipitated solid was collected by filtration to obtain a crude product of ampicillin (HPLC purity: 96%). 5% by mass of sodium bicarbonate water (150 g) was added to the obtained crude product of ampicillin and dissolved. Next, the operation of adding ethyl acetate (100 mL) to wash the water phase was repeated twice, 1 N hydrochloric acid was added to the aqueous layer to adjust the pH to 4.5 to 5.0, and the precipitated solid was collected by filtration to obtain a purified product of ampicillin (yield: 9.7 g, yield: 80%, HPLC purity: 98%). In this flow-type reaction, no flow obstacle such as clogging occurred in each flow channel and the merging section.

The “HPLC purity” is calculated by the following expression by creating a calibration curve using a standard product of ampicillin under the following operating conditions using the following high performance liquid chromatography (HPLC) device.
Apparatus: prominence series (manufactured by Shimadzu Corporation)
Detector: UV/VIS light absorption detector SPD-20A, 220 nm
Column: Waters Atlantis T3 3 um 4.6 mmφ×250 mm
Column oven: 30° C.
Mobile phase A: water, 0.1% phosphoric acid
Mobile phase B: acetonitrile, 0.1% phosphoric acid
Mobile phase concentration gradient: 0.01 min (B 10%), 10.00 min (B 80%), 15.00 min (B 80%), 15.01 min (B 10%), 17.00 min (B 10%)

HPLC purity (%)=100×[yield of ampicillin based on calibration curve]/[theoretical yield of ampicillin calculated based on amount of charge]

In the above-described flow-type reaction, for example, by using a compound in which the benzene ring of phenylglycine is replaced with a 1,4-cyclohexadiene ring instead of phenylglycine, or by using a compound in which 7-ADCA or a methyl thereof is replaced with a chlorine atom instead of 6-APA, various useful β-lactam compounds having antibacterial activity, such as epicillin, cephalexin, cefradine, and cefaclor, can be continuously and efficiently synthesized by the flow-type reaction in the same manner as the above-described ampicillin.

The present invention has been described using the embodiments. However, unless specified otherwise, any of the details of the above description is not intended to limit the present invention and can be construed in a broad sense within a range not departing from the concept and scope of the present invention disclosed in the accompanying claims.

What is claimed is:

1. A method for producing a β-lactam compound, the method comprising:
introducing, into different flow channels, each of
a reaction solution (i) obtained by dissolving a metal salt (y) represented by Formula (2) generated by introducing, in a solution obtained by dissolving a metal salt (x) represented by Formula (1), a protective group into an amino group of the metal salt (x),
a solution (ii) obtained by dissolving an acid halide, and
a solution (iii) obtained by dissolving a β-lactam compound (a) having an amino group, and causing each solution to flow in each flow channel;
merging the reaction solution (i) and the solution (ii) to cause the metal salt (y) and the acid halide to react with each other in a merged solution (M1) while the merged solution (M1) flows downstream to generate a mixed acid anhydride; and
merging the merged solution (M1) and the solution (iii) to cause the mixed acid anhydride and the β-lactam compound (a) having an amino group to react with each other in a merged solution (M2) while the merged solution (M2) flows downstream to generate a β-lactam compound (b) having an amide bond, 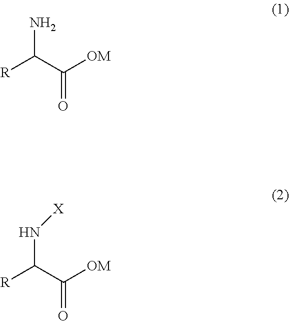
in the formulae, R represents a cyclic hydrocarbon group, M represents an alkali metal, and X represents a protective group.

2. The method for producing a β-lactam compound according to claim 1,
wherein the reaction solution (i) and the solution (ii) are merged at −40° C. to 0° C.

3. The method for producing a β-lactam compound according to claim 2,
wherein the merged solution (M1) and the solution (iii) are merged at −40° C. to 0° C.

4. The method for producing a β-lactam compound according to claim 3,
wherein the solution (iii) contains 5% to 50% by mass of water.

5. The method for producing a β-lactam compound according to claim 4,
wherein the solution (iii) contains a base.

6. The method for producing a β-lactam compound according to claim 5,
wherein the protective group is a 3-ethoxy-1-methyl-3-oxo-1-propenyl group and/or a 3-methoxy-1-methyl-3-oxo-1-propenyl group.

7. The method for producing a β-lactam compound according to claim 6,
wherein the acid halide includes a carboxylic acid halide.

8. The method for producing a β-lactam compound according to claim 7,
wherein a reaction between the metal salt (y) and the carboxylic acid halide in the merged solution (M1) is performed in the presence of a basic catalyst.

9. The method for producing a β-lactam compound according to claim 8,
wherein the basic catalyst has a pyridine skeleton.

10. The method for producing a β-lactam compound according to claim 9,
wherein a molar amount of the basic catalyst is set to 0.001 to 0.1 with respect to a molar amount of 1 of the β-lactam compound (a) having an amino group.

11. The method for producing a β-lactam compound according to claim 10,
wherein the carboxylic acid halide includes a carboxylic acid chloride.

12. The method for producing a β-lactam compound according to claim 1,
wherein a hydrocarbon ring of the cyclic hydrocarbon group is a benzene ring or a 1,4-cyclohexadiene ring.

13. The method for producing a β-lactam compound according to claim 12,
wherein M is sodium or potassium.

14. The method for producing a β-lactam compound according to claim 12,
wherein the β-lactam compound (a) having an amino group is 6-aminopenicillanic acid or 7-amino-desacetoxycephalosporanic acid.

15. The method for producing a β-lactam compound according to claim 14,
wherein the β-lactam compound (b) is ampicillin.
